(19) United States
(12) Patent Application Publication
(10) Pub. No.: US 2026/0020777 A1
(43) Pub. Date: Jan. 22, 2026

(54) Obtaining Biometric Information of a User Based on a Ballistocardiogram Signal Obtained When a Mobile Computing Devie is Held Against the Head of the User

(71) Applicant: Google LLC, Mountain View, CA (US)

(72) Inventor: Dongeek Shin, San Jose, CA (US)

(21) Appl. No.: 18/995,675

(22) PCT Filed: Jul. 18, 2022
(86) PCT No.: PCT/US2022/037447

§ 371 Date: Jan. 16, 2025

(51) Int. Cl. A61B 5/11; A61B 5/00

(52) U.S. Cl. CPC A61B 5/1102; A61B 5/1126; A61B 5/6898; A61B 5/7257; A61B 5/746; A61B 5/7475; A61B 2562/0219

(57) ABSTRACT

A mobile computing device includes one or more memories to store one or more instructions. an inertial measurement unit. and one or more processors. The one or more processors execute the one or more instructions stored in the one or more memories to: control the inertial measurement unit to detect one or more motion signals generated when the mobile computing device is held against a head of a user of the mobile computing device. determine a ballistocardiogram signal based on the one or more motion signals detected by the inertial measurement unit. obtain, based on the ballistocardiogram signal. biometric information of the user, and output the biometric information of the user.

### FIELD

The disclosure relates generally to mobile computing devices. More particularly, the disclosure relates to mobile computing devices which are used to obtain biometric information of a user based on a ballistocardiogram signal obtained when the mobile computing device is held against a head of the user of the mobile computing device.

### BACKGROUND

Some wearable computing devices, such as a fitness watch, are capable of measuring human biometric information passively (e.g., without requiring the user to use their cognitive load to actively press a button to start some measurement). However, such devices are separate accessories that must be worn on a body part of the user.

Some mobile phones are capable of active heart rate monitoring by using a standard optical solution where a user places a finger on a camera to take a biometric measurement.

### SUMMARY

Aspects and advantages of embodiments of the disclosure will be set forth in part in the following description, or can be learned from the description, or can be learned through practice of the example embodiments.

In an example embodiment, a mobile computing device, such as a mobile phone or a smartphone, is provided. The mobile computing device includes one or more memories configured to store one or more instructions, an inertial measurement unit, and one or more processors configured to execute the one or more instructions stored in the one or more memories to: control the inertial measurement unit to detect one or more motion signals generated when the mobile computing device is held against a head of a user of the mobile computing device, determine a ballistocardiogram signal based on the one or more motion signals detected by the inertial measurement unit, obtain, based on the ballistocardiogram signal, biometric information of the user, and output the biometric information of the user.

In some implementations, the one or more processors are configured to automatically execute the one or more instructions stored in the one or more memories to control the inertial measurement unit to detect the one or more motion signals, in response to a telephone call being conducted using the mobile computing device.

In some implementations, the one or more motion signals are generated based on blood vessel volume changes in a temple region of the head of the user.

In some implementations, the mobile computing device includes an output device configured to provide, during the telephone call, an indication that the mobile computing device is performing a process to obtain the biometric information of the user. In some implementations, the indication includes at least one of a sound provided by the output device or haptic feedback provided by the output device.

In some implementations, the mobile computing device includes a ballistocardiographic autoencoder and a spectral analyzer. The one or more processors are configured to determine the ballistocardiogram signal by controlling the ballistocardiographic autoencoder to convert the one or more motion signals detected by the inertial measurement unit to the ballistocardiogram signal, and the one or more processors are configured to obtain the biometric information of the user by controlling the spectral analyzer to: perform a Fast Fourier transform with respect to the ballistocardiogram signal over a predetermined window of the ballistocardiogram signal, detect peaks, with respect to the ballistocardiogram signal, during the predetermined window; and obtain the biometric information of the user based on the peaks which are detected during the predetermined window.

In some implementations, the ballistocardiographic autoencoder is configured to convert the one or more motion signals detected by the inertial measurement unit to the ballistocardiogram signal using a machine learning resource which predicts the ballistocardiogram signal based on training data that maps a relationship between previous motion signals and corresponding ground truth ballistocardiogram signals.

In some implementations, the spectral analyzer is configured to determine whether the ballistocardiogram signal satisfies one or more predetermined conditions before determining the biometric information of the user based on the ballistocardiogram signal. The one or more predetermined conditions may be associated with at least one of a noise level of the ballistocardiogram signal, a motion of the user, or a sparsity level of the ballistocardiogram signal.

In some implementations, when a first predetermined condition among the one or more predetermined conditions relates to the noise level of the ballistocardiogram signal, the spectral analyzer is configured to determine the ballistocardiogram signal satisfies the first predetermined condition when the noise level associated with the ballistocardiogram signal is less than a threshold noise level. In some implementations, when a second predetermined condition among the one or more predetermined conditions relates to the motion of the user, the spectral analyzer is configured to determine the ballistocardiogram signal satisfies the second predetermined condition when the user is determined to be at rest. In some implementations, when a third predetermined condition among the one or more predetermined conditions relates to the sparsity level of the ballistocardiogram signal, the spectral analyzer is configured to determine the ballistocardiogram signal satisfies the third predetermined condition when the sparsity level of the ballistocardiogram signal is greater than a threshold sparsity level.

In some implementations, the spectral analyzer is configured to: determine a difference between a maximum energy level of the ballistocardiogram signal and a median energy level of the ballistocardiogram signal during the predetermined window, and determine the ballistocardiogram signal satisfies the third predetermined condition when the difference between the maximum energy level and the median energy level is greater than the threshold sparsity level.

In some implementations, the inertial measurement unit includes one or more accelerometers and one or more gyroscopes to detect the one or more motion signals generated when the mobile computing device is held against the head of the user. The one or more motion signals may include a six-dimensional motion signal, and the ballistocardiographic autoencoder is configured to convert the six-dimensional motion signal to a one-dimensional ballistocardiogram signal.

In some implementations, the mobile computing device is a mobile phone or a smartphone.

In some implementations, the biometric information of the user includes at least one of a heart rate of the user or a heart rate variability of the user.

In some implementations, the one or more processors are configured to output the biometric information of the user by at least one of: storing the biometric information of the user in at least one of a database, the one or more memories, or one or more memories of an external computing device, presenting the biometric information of the user on a display of the mobile computing device, or generating a report which summarizes the biometric information of the user.

In some implementations, the mobile computing device includes an output device and the one or more processors are configured to analyze the biometric information of the user to determine whether the biometric information of the user indicates a presence of an abnormality or an irregularity in the biometric information of the user. The one or more processors may be configured to control the output device to provide at least one of a warning, an alert, or a notification to the user in response to determining the biometric information of the user indicates the presence of the abnormality or the irregularity in the biometric information of the user.

In some implementations, the mobile computing device includes an input device configured to receive a request from the user to obtain the biometric information of the user, and an output device configured to provide information regarding a measurement time period for which the mobile computing device is to be held against the head of the user of the mobile computing device.

In an example embodiment, a computer-implemented method is provided. The computer-implemented method includes detecting, by an inertial measurement unit of a mobile computing device, one or more motion signals generated when the mobile computing device is held against a head of a user of the mobile computing device, converting the one or more motion signals detected by the inertial measurement unit to a ballistocardiogram signal, obtaining, based on the ballistocardiogram signal, biometric information of the user, and outputting the biometric information of the user.

In some implementations, the method includes automatically controlling the inertial measurement unit to detect the one or more motion signals in response to a telephone call being conducted using the mobile computing device.

In some implementations, the method includes converting the one or more motion signals detected by the inertial measurement unit to the ballistocardiogram signal includes using a machine learning resource which predicts the ballistocardiogram signal based on training data that maps a relationship between previous motion signals and corresponding ground truth ballistocardiogram signals.

In an example embodiment, a non-transitory computer-readable medium which stores instructions that are executable by one or more processors of a mobile computing device is provided. The non-transitory computer-readable medium stores instructions which are executable by one or more processors of a mobile computing device. The instructions include: instructions to cause the one or more processors to control an inertial measurement unit of the mobile computing device to detect one or more motion signals generated when the mobile computing device is held against a head of a user of the mobile computing device, instructions to cause the one or more processors to convert the one or more motion signals detected by the inertial measurement unit to a ballistocardiogram signal, instructions to cause the one or more processors to obtain, based on the ballistocardiogram signal, biometric information of the user, and instructions to cause the one or more processors to output the biometric information of the user.

The non-transitory computer-readable medium may store additional instructions to execute other aspects and operations of the mobile computing device and computer-implemented method as described herein.

In an example embodiment, a mobile device is provided. The mobile device, which includes an inertial measurement unit, is used for determining a ballistocardiogram signal based on one or more motion signals detected by the inertial measurement unit to obtain biometric information of a user holding the mobile device against a head of the user, for example by performing the computer-implemented method disclosed herein.

These and other features, aspects, and advantages of various embodiments of the disclosure will become better understood with reference to the following description, drawings, and appended claims. The accompanying drawings, which are incorporated in and constitute a part of this specification, illustrate examples of the disclosure and, together with the description, serve to explain the related principles.

### BRIEF DESCRIPTION OF THE DRAWINGS

Detailed discussion of example embodiments directed to one of ordinary skill in the art is set forth in the specification, which makes reference to the appended drawings, in which:

FIG. 1 is an example system including block diagrams of a mobile computing device and one or more external computing devices, according to one or more examples of the disclosure:

FIG. 2 is an example illustration of a user holding a mobile computing device against the head of the user, according to one or more examples of the disclosure:

FIG. 3 is an example illustration including a block diagram of a biometric measurement application, according to one or more examples of the disclosure:

FIG. 4 is an example flow diagram for obtaining biometric information of a user, according to one or more examples of the disclosure: and

FIG. 5 is a flow diagram of an example, non-limiting computer-implemented method according to one or more examples of the disclosure.

### DETAILED DESCRIPTION

Reference now will be made to embodiments of the disclosure, one or more examples of which are illustrated in the drawings. Each example is provided by way of explanation of the disclosure and is not intended to limit the disclosure. In fact, it will be apparent to those skilled in the art that various modifications and variations can be made to the disclosure without departing from the scope or spirit of the disclosure. For instance, features illustrated or described as part of one embodiment can be used with another embodiment to yield a still further embodiment. Thus, it is intended that the disclosure covers such modifications and variations as come within the scope of the appended claims and their equivalents.

Terms used herein are used to describe the example embodiments and are not intended to limit and/or restrict the disclosure. The singular forms “a,” “an” and “the” are intended to include the plural forms as well, unless the context clearly indicates otherwise. In this disclosure, terms such as “including”, “having”, “comprising”, and the like are used to specify features, numbers, steps, operations, elements, components, or combinations thereof, but do not preclude the presence or addition of one or more of the features, elements, steps, operations, elements, components, or combinations thereof.

It will be understood that, although the terms first, second, third, etc., may be used herein to describe various elements, the elements are not limited by these terms. Instead, these terms are used to distinguish one element from another element. For example, without departing from the scope of the disclosure, a first element may be termed as a second element, and a second element may be termed as a first element.

The term “and/or” includes a combination of a plurality of related listed items or any item of the plurality of related listed items. For example, the scope of the expression or phrase “A and/or B” includes the item “A”, the item “B”, and the combination of items “A and B”.

In addition, the scope of the expression or phrase “at least one of A or B” is intended to include all of the following: (1) at least one of A, (2) at least one of B, and (3) at least one of A and at least one of B. Likewise, the scope of the expression or phrase “at least one of A, B, or C” is intended to include all of the following: (1) at least one of A, (2) at least one of B, (3) at least one of C, (4) at least one of A and at least one of B, (5) at least one of A and at least one of C, (6) at least one of B and at least one of C, and (7) at least one of A, at least one of B, and at least one of C.

Examples of the disclosure are directed to a mobile computing device, for example, a mobile phone or a smartphone, that can be used to measure biometric information of a user. For example, the mobile computing device may be configured to measure biometric information of the user when the mobile computing device is held against a head of the user (e.g., a temple region of the head). In more detail, the mobile computing device includes an inertial measurement unit (IMU) which is configured to detect and amplify small above-skin motion signals caused by blood vessel volume changes for every pump of blood from the heart of the user throughout the body. The motion signals detected by the IMU are transferred to a ballistocardiographic autoencoder which is configured to output a realistic ballistocardiogram (BCG) signal based on the motion signals detected by the IMU. The BCG signal corresponds to the small vibrations around the temple of the user which correlates to heart beats. That is, the BCG signal reflects a reaction (e.g., a displacement, velocity, and/or acceleration) of a part of the body resulting from cardiac ejection of blood. A spectral analyzer analyzes the BCG signal to output biometric information about the user, such as heart rate (HR), heart rate variability (HRV), etc.

In some implementations the biometric information of the user can be measured by the mobile computing device in an active manner or a passive manner. For example, a user may actively open a biometric related application and place the mobile computing device against their head and intentionally request that a biometric measurement be obtained. In some implementations, the mobile computing device may automatically measure biometric information of the user (without a specific request from the user) in a passive manner. As an example, when the mobile computing device is used for a phone call and the user places the mobile computing device against their head the mobile computing device may be configured to automatically obtain a biometric measurement of the user.

Measuring human biometric information passively (e.g., without requiring the user to use their cognitive load to actively press a button to start some measurement) may be performed using a wearable computing device, such as a fitness watch. However, wearable computing devices are separate accessories that must be worn on a body part of the user.

According to examples of the disclosure, a mobile computing device, such as a mobile phone or smartphone, can passively measure biometric information of the user. In addition, the mobile computing device may be a device that the user already carries with them, for example to make telephone calls.

For example, the mobile computing device may be configured to, when a user makes or receives a telephone call and places the mobile computing device against their head (e.g., a temple region of the head), passively measure biometric information of the user. For example, when the mobile computing device makes contact with the temple region of the head, high resolution ballistocardiogram (BCG) recordings may be obtained and processed by the mobile computing device via the IMU, ballistocardiographic autoencoder, and spectral analyzer. For example, the IMU opportunistically detects and amplifies small above-skin motion signals caused by blood vessel volume changes (due to changes in arterial content) during a user's phone call session. The above-skin motion signals may be measured at the temple region of the head of the user, for example. The motion signals are received by the ballistocardiographic autoencoder which outputs a realistic BCG signal. For example, the ballistocardiographic autoencoder may predict a BCG signal based on a machine learning algorithm (e.g., using a custom neural network) and training data. The spectral analyzer analyzes the BCG signal predicted by the ballistocardiographic autoencoder and outputs biometric information such as the heart rate of the user and heart rate variability of the user. For example, the biometric information of the user can be recorded in a database and displayed to the user, for example in the form of a vital signs report.

According to examples of the disclosure, the motion signals measured by the IMU may include a six-dimensional IMU signal that includes information from one or more accelerometers (three-dimensions) and one or more gyroscopes (three-dimensions). For example, the motion signals may be sampled at a sample rate of 100 Hertz. The mobile computing device is placed against the head of the user (e.g., the temple region of the head) so that the IMU can accurately detect and measure blood vessel volume change information.

The motion signals measured by the IMU are transmitted to the ballistocardiographic autoencoder to be converted to a BCG signal for cardiovascular analytics to be performed. The IMU motion signals may be converted to a BCG signal based on a mapping relationship between previously measured IMU motion signals and corresponding BCG signals which are experimentally obtained and used as training data for training a machine learning resource such as a custom neural network (e.g., an unsupervised neural network). For example, the ballistocardiographic autoencoder is configured to map the 6-dimensional IMU motion signals into a 1-dimensional BCG signal. The custom neural network (e.g., an unsupervised neural network) may include, as training data, IMU data (e.g., raw IMU motion signals) which is collected from users during a phone call session and a ground truth BCG signal data collected from a BCG measurement device such as a piezoelectric sensor (e.g. a piezo band) which can be placed around the head or chest of the user during the phone call session. In some implementations, other BCG measurement devices may be used to obtain a ground truth BCG signal such as a strain gauge, fiberoptic sensor, etc. The custom neural network may be trained to predict a BCG signal (e.g., through regressive modeling) according to received IMU motion signals based on the training data and a machine learning algorithm that is derived from the training which occurs offline. Thus, a generalized mapping can occur between a 6-dimensional IMU signal obtained from the IMU and a 1-dimensional BCG signal using the ballistocardiographic autoencoder.

The 1-dimensional BCG signal output by the ballistocardiographic autoencoder is analyzed by the spectral analyzer to obtain biometric information of the user. For example, in some implementations the spectral analyzer is configured to take a finite buffer of the BCG signal, window the finite buffer in a manner to ensure infinity-spectral leakage does not occur, take a Fast Fourier transform of the signal over the window, and perform some smoothed-variant of peak finding (e.g., using a peak detector) to locate a heart rate in beats per minute based on where a frequency has a maximum energy level in the frequency domain.

The spectral analyzer is also configured to analyze the BCG signal to ensure or verify that the obtained BCG signal satisfies one or more predetermined conditions before the spectral analyzer proceeds with measuring the biometric information of the user from the BCG signal. For example, certain characteristics of the BCG signal may not satisfy the one or more predetermined conditions when the user is not at rest. For example, when the user is moving the BCG signal may become contaminated with motion artifacts and biometric measurements of the BCG signal may be erroneous and/or inaccurate. For example, the one or more predetermined conditions may not be satisfied when a noise level of the BCG signal exceeds a predetermined threshold level. For example, the one or more predetermined conditions may not be satisfied when a sparsity level of the BCG signal is less than a threshold level. For example, the sparsity level may be determined based on spectral features relating to peaks of the BCG signal relative to other portions of the BCG signal. For example, the sparsity level may be determined based on a difference between a maximum energy level of the BCG signal and a median energy level of the BCG signal. That is, the sparsity level may be acceptable when the difference between the maximum energy level and the median energy level is greater than a threshold level, and unacceptable when the difference between the maximum energy level and the median energy level is less than the threshold level. The spectral analyzer may continuously perform checks of spectral features of the BCG signal which is obtained during a phone call to determine whether the one or more predetermined conditions are satisfied, and when the one or more predetermined conditions are satisfied the spectral analyzer may be configured to measure the biometric information of the user, for example by determining a heart rate and/or heart rate variability of the user based on the BCG signal.

In some implementations, the spectral analyzer may analyze the BCG signal to ensure or verify that the obtained BCG signal satisfies one or more predetermined conditions according to a machine learning resource. For example, the machine learning resource of the spectral analyzer may be trained to determine whether the one or more predetermined conditions are satisfied by training a shallow, fully-connected neural network classifier on the spectral features of the BCG signal to perform a binary classification of (1) the one or more predetermined conditions are satisfied and it is a good time to proceed with measuring the biometric information of the user, or (2) the one or more predetermined conditions are not satisfied and it is not a good time to proceed with measuring the biometric information of the user. The spectral analyzer proceeds to measuring the biometric information of the user such as the heart rate or heart rate variability when the machine learning resource classifies the spectral features of the BCG signal according to the first classification. In some implementations, if the spectral analyzer does not proceed with measuring the biometric information of the user when the machine learning resource classifies the spectral features of the BCG signal according to the second classification, the output device of the mobile computing device may be configured to provide an indication to the user. For example, if the spectral features of the BCG signal are classified according to the second classification due to movement of the user, the output device may provide an indication to the user to remain still (e.g., via a speaker, display device, etc.).

The spectral analyzer is configured to measure or compute the biometric information of the user based on the BCG signal. The mobile computing device may be configured to store relevant biometric information and display the biometric information. In some implementations, the biometric information may be displayed or provided to the user according to a time and/or date that the biometric information was obtained (e.g., during a phone call session). A report may be generated by the mobile computing device or a health-related application which summarizes the biometric information obtained. For example, the report may include a monthly summary, average resting heart parameters for the user, etc. For example, the report may include information such as a graph that is generated by interpolating (e.g., via exponential averaging and interpolation) the obtained biometric information of the user so that the user can view their heart rate as a continuous function of time.

According to additional aspects of the disclosure, the mobile computing device may include a virtual assistant and/or an output device to guide or assist a user with taking a biometric measurement. For example, the output device may provide a countdown via a speaker for the user so that the user holds the mobile computing device against their head for a sufficient duration of time (e.g., 20 seconds to 30 seconds) in order to take a biometric measurement. For example, when the user makes or receives a phone call, the output device may provide an indication to the user (e.g., as haptic feedback through a haptic device or a sound output by a speaker) which serves as a reminder that the mobile computing device is taking a biometric measurement during the phone call. Therefore, the user may be more likely to hold the phone against their head during the phone call to obtain an accurate biometric measurement. In some implementations, if a phone call is less than a predetermined duration of time (e.g., less than 20 seconds to 30 seconds) or a measurement period during the phone call is less than the predetermined duration of time, the BCG biometric measurement application may discard any data or biometric measurements obtained during the brief phone call.

According to additional aspects of the disclosure, the mobile computing device may also include a proximity sensor which is configured to sense a distance between the mobile computing device and the head of the user. If the proximity sensor detects that a distance between the mobile computing device and the head is less than a predetermined threshold, the mobile computing device may be configured to execute or enable a BCG biometric measurement application so that biometric information of the user can be obtained when the mobile computing device is pressed against the head of the user. If the proximity sensor detects that a distance between the mobile computing device and the head is greater than the predetermined threshold, the mobile computing device may be configured to terminate or disable the BCG biometric measurement application. In addition, the output device of the mobile computing device may provide an indication to the user to move the mobile computing device to be in contact with the head of the user (e.g., via a message played by a speaker, by a message presented on a display device of the mobile computing device, etc.). In some implementations, instead of a proximity sensor other devices such as a camera or LIDAR may be used to measure a distance between the mobile computing device and the head of the user.

Example aspects of the disclosure provide several technical effects, benefits, and/or improvements in computing technology and the technology of mobile computing devices and health monitoring devices. For example, according to one or more examples of the disclosure, passive measurements of biometric information of the user may be obtained accurately without the user having to actively press a button for any sort of measurement and/or without the user having to open a biometric measurement application. Instead, according to examples of the disclosure a passive BCG measurement application can opportunistically measure biometric information of the user during a phone call (e.g., with appropriate user permissions and consent previously given for such measurements to be taken, for a cardiovascular health database to be built in the background, for transmitting such information to other devices such as a server computing system, etc.). The mobile computing device also leverages existing sensors (e.g., the IMU) of the mobile computing device to obtain the biometric information of the user so additional sensors are not needed.

Known heart rate monitoring on mobile phones use a standard optical solution where a user places a finger on a camera to take a biometric measurement. In contrast, according to examples of the disclosure a passive BCG measurement application is executed in connection with (or in response to) a phone call session being conducted and a biometric information of the user is computed from a BCG signal obtained while the mobile computing device is in contact with the user's head.

Greater amounts of biometric information of the user can be collected due to the passive nature of BCG biometric measurement application, resulting in a fuller and more accurate picture of the user's health. A comprehensive cardiovascular report can be generated and may be referred to by medical professionals for assessing a patient's health during visits to a medical office. For example, passive health monitoring allows for a health database to be built up in the background over time. In some implementations, the mobile computing device may be configured to output a warning or alert to the user by interpreting trends and detecting abnormal behaviors in the health data. Therefore, the mobile computing device can be intelligent enough to provide users preventative information and warnings by tracking and monitoring the biometric information obtained via the BCG biometric measurement application.

In various examples disclosed herein, machine learning resources may learn through one or more various machine learning techniques (e.g., by training a neural network or other machine-learned model) to predict or model BCG signals, based on motion signals output by an inertial measurement unit, where the motion signals are generated or caused by blood vessel volume changes for every pump of blood from the heart of a user throughout their body. For example, ground truth BCG signals which correspond to IMU motion signals can be stored and used as training data to train (e.g., via supervised or unsupervised training techniques) one or more machine-learned models to, after training, generate predictions or models of a BCG signal for received IMU motion signals. In such a way, system performance is improved by not requiring a separate BCG measurement device and by not requiring a user to wear a BCG measurement device such as a chest piezo band or a head piezo band.

In various examples disclosed herein, machine learning resources may learn through one or more various machine learning techniques (e.g., by training a neural network or other machine-learned model) to determine (e.g., with a specified confidence level, with a probability above a threshold level, etc.), whether a BCG signal is of a sufficient quality for biometric information to be measured based on the BCG signal, or is deficient and should be discarded or ignored. For example, data descriptive of BCG signals which are “clean” signals and satisfy various predetermined conditions can be stored and used as training data to train (e.g., via supervised or unsupervised training techniques) one or more machine-learned models to, after training, generate predictions which assist in determining whether the BCG signal is of sufficient quality for biometric information to be measured based on the BCG signal, or is deficient and should be discarded or ignored. In such a way, system performance is improved with more accurate and reliable biometric information measurements. Furthermore, processing, memory, and network resources of a computing system (e.g., a mobile computing device, external computing device, or combinations thereof) are conserved by not measuring biometric information of BCG signals which are deficient.

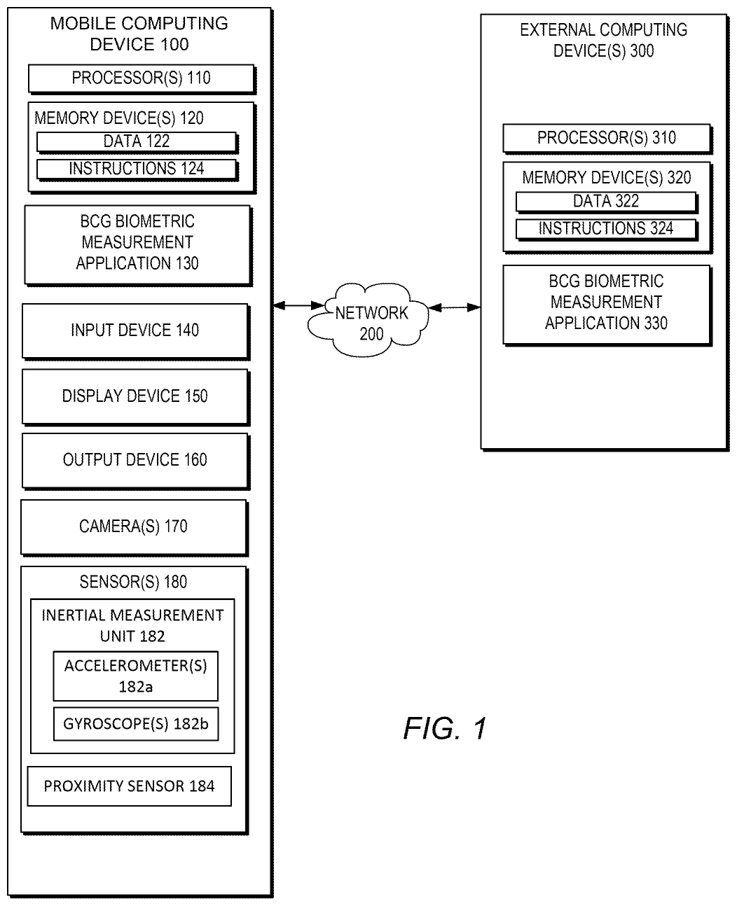

Referring now to the drawings, FIG. 1 illustrates an example system including block diagrams of a mobile computing device and one or more external computing devices, according to one or more examples of the disclosure. In FIG. 1, the example system includes a mobile computing device 100 and one or more external computing devices 300 which are connected with one another over a network 200. Any communications interfaces suitable for communicating via the network 200 (such as a network interface card) may be utilized as appropriate or desired by the mobile computing device 100 and one or more external computing devices 300.

The one or more external computing devices 300 may include a personal computer, a smartphone, a laptop, a tablet computer, and the like. The one or more external computing devices 300 may also include a server computing system. The server computing system can include a server, or a combination of servers (e.g., a web server, application server, etc.) in communication with one another, for example in a distributed fashion.

According to some implementations of the disclosure, the mobile computing device 100 may communicate with the one or more external computing devices 300 to share biometric information of the user (e.g., to store the biometric information in a database of a server computing system, a medical service provider, a laptop, etc.).

According to some implementations of the disclosure, the mobile computing device 100 may communicate with the one or more external computing devices 300 to obtain biometric information of the user. For example, the one or more external computing devices 300 may be configured to receive one or more motion signals from the mobile computing device 100, where the one or more motion signals are detected by the inertial measurement unit 182 of the mobile computing device 100 when the mobile computing device 100 is held against a head of a user of the mobile computing device 100. The one or more external computing devices 300 may be further to configured determine a ballistocardiogram signal based on the one or more motion signals and to obtain, based on the ballistocardiogram signal, biometric information of the user. The one or more external computing devices 300 may be further to configured to transmit the biometric information to the mobile computing device 100 via the network 200. The one or more external computing devices 300 may include features such as one or more processors 310, one or more memory devices 320, and one or more ballistocardiogram (BCG) biometric measurement applications 330, which are similar to corresponding features of the mobile computing device 100, as discussed in more detail below:

For example, the network 200 may include any type of communications network such as a local area network (LAN), wireless local area network (WLAN), wide area network (WAN), personal area network (PAN), virtual private network (VPN), or the like. For example, wireless communication between elements of the examples described herein may be performed via a wireless LAN, Wi-Fi, Bluetooth, ZigBee, Wi-Fi direct (WFD), ultra wideband (UWB), infrared data association (IrDA), Bluetooth low energy (BLE), near field communication (NFC), a radio frequency (RF) signal, and the like. For example, wired communication between elements of the examples described herein may be performed via a pair cable, a coaxial cable, an optical fiber cable, an Ethernet cable, and the like. Communication over the network can use a wide variety of communication protocols (e.g., TCP/IP, HTTP, SMTP, FTP), encodings or formats (e.g., HTML, XML), and/or protection schemes (e.g., VPN, secure HTTP, SSL).

The mobile computing device 100 may be a mobile phone or a smartphone, for example. The mobile computing device 100 may include one or more processors 110, one or more memory devices 120, a BCG biometric measurement application 130, an input device 140, a display device 150, an output device 160, one or more cameras 170, and one or more sensors 180. Each of the components of the mobile computing device 100 may be operatively connected with one another via a system bus. For example, the system bus may be any of several types of bus structures that may further interconnect to a memory bus (with or without a memory controller), a peripheral bus, and/or a local bus using any of a variety of commercially available bus architectures. The one or more external computing devices 300 may include one or more processors 310, one or more memory devices 320, and one or more BCG biometric measurement applications 330. Each of the features of the one or more external computing devices 300 may be operatively connected with one another via a system bus. For example, the system bus may be any of several types of bus structures that may further interconnect to a memory bus (with or without a memory controller), a peripheral bus, and/or a local bus using any of a variety of commercially available bus architectures.

For example, the one or more processors 110, 310 can be any suitable processing device that can be included in a mobile computing device 100 or in one of the one or more external computing devices 300. For example, such a processor 110, 310 may include one or more of a processor, processor cores, a controller and an arithmetic logic unit, a central processing unit (CPU), a graphics processing unit (GPU), a digital signal processor (DSP), an image processor, a microcomputer, a field programmable array, a programmable logic unit, an application-specific integrated circuit (ASIC), a microprocessor, a microcontroller, etc., and combinations thereof, including any other device capable of responding to and executing instructions in a defined manner. The one or more processors 110, 310 can be a single processor or a plurality of processors that are operatively connected, for example in parallel.

The one or more memory devices 120, 320 can include one or more non-transitory computer-readable storage mediums, such as such as a Read Only Memory (ROM), Programmable Read Only Memory (PROM), Erasable Programmable Read Only Memory (EPROM), and flash memory, a USB drive, a volatile memory device such as a Random Access Memory (RAM), an internal or external hard disk drive (HDD), floppy disks, a blue-ray disk, or optical media such as CD ROM discs and DVDs, and combinations thereof. However, examples of the one or more memory devices 120, 320 are not limited to the above description, and the one or more memory devices 120, 320 may be realized by other various devices and structures as would be understood by those skilled in the art.

For example, the one or more memory devices 120 can store instructions, that when executed, cause the one or more processors 110 to control the inertial measurement unit 182 of the mobile computing device 100 to detect one or more motion signals generated when the mobile computing device 100 is held against a head of a user of the mobile computing device 100, as described according to examples of the disclosure. For example, the one or more memory devices 120 can store instructions that, when executed, cause the one or more processors 110 to convert the one or more motion signals detected by the inertial measurement unit 182 to a ballistocardiogram signal, as described according to examples of the disclosure. For example, the one or more memory devices 120 can store instructions, that when executed, cause the one or more processors 110 to obtain, based on the ballistocardiogram signal, biometric information of the user and to output the biometric information of the user, as described according to examples of the disclosure.

For example, the one or more memory devices 320 can store instructions, that when executed, cause the one or more processors 310 to receive from the mobile computing device 100 one or more motion signals detected by the inertial measurement unit 182 when the mobile computing device 100 is held against a head of a user of the mobile computing device 100, as described according to examples of the disclosure. For example, the one or more memory devices 320 can store instructions, that when executed, cause the one or more processors 310 to convert the one or more motion signals received from the mobile computing device 100 to a ballistocardiogram signal, as described according to examples of the disclosure. For example, the one or more memory devices 320 can store instructions, that when executed, cause the one or more processors 310 to obtain, based on the ballistocardiogram signal, biometric information of the user and to output the biometric information of the user, as described according to examples of the disclosure.

The one or more memory devices 120 can also include data 122 and instructions 124 that can be retrieved, manipulated, created, or stored by the one or more processors 110. In some examples, such data can be accessed and used as input to obtain and output the biometric information of the user, as described according to examples of the disclosure. The one or more memory devices 320 can also include data 322 and instructions 324 that can be retrieved, manipulated, created, or stored by the one or more processors 310. In some examples, such data can be accessed and used as input to obtain and output the biometric information of the user, as described according to examples of the disclosure.

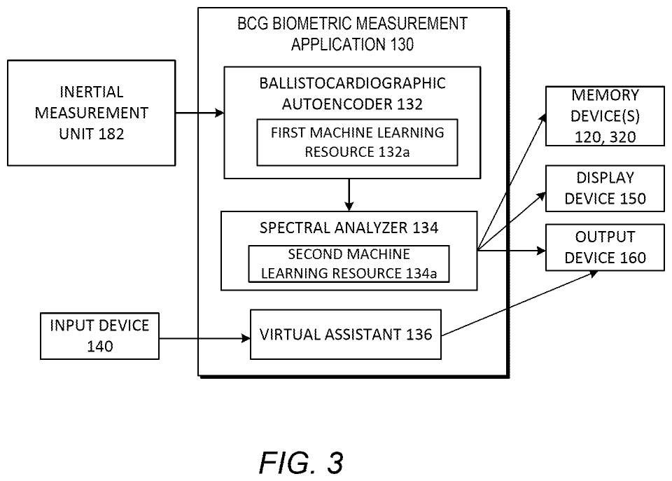

The BCG biometric measurement application 130 can include any biometric application which allows or is capable of allowing a user to measure biometric information of a user using the mobile computing device 100, based on a BCG signal. In some implementations, the BCG biometric measurement application 130 includes a ballistocardiographic autoencoder 132, spectral analyzer 134, and virtual assistant 136 (see FIG. 3). The BCG biometric measurement application 130 may be executed in an active manner or a passive manner. For example, a user may actively execute the BCG biometric measurement application 130 by providing an input to the mobile computing device 100 to take a biometric measurement. For example, the BCG biometric measurement application 130 may be passively executed when a user makes or receives a phone call. The BCG biometric measurement application 330 of the one or more external computing devices 300 can be used in connection with the mobile computing device 100 to perform at least some similar operations of the BCG biometric measurement application 130 to obtain and output biometric information of a user, and may include similar features of the ballistocardiographic autoencoder 132, spectral analyzer 134, and virtual assistant 136, as shown in FIG. 3.

The mobile computing device 100 may include an input device 140 configured to receive an input from a user and may include, for example, one or more of a keyboard (e.g., a physical keyboard, virtual keyboard, etc.), a mouse, a joystick, a button, a switch, an electronic pen or stylus, a gesture recognition sensor (e.g., to recognize gestures of a user including movements of a body part), an input sound device or voice recognition sensor (e.g., a microphone to receive a voice command), a track ball, a remote controller, a portable (e.g., a cellular or smart) phone, and so on. The input device 140 may also be embodied by a touch-sensitive display device having a touchscreen capability, for example. The input device 140 may be used by a user of the mobile computing device 100 to provide an input to request to take a biometric measurement, to provide an input to execute the BCG biometric measurement application 130 (see FIG. 3), to transmit biometric information of the user to the one or more external computing devices 300, etc. For example, the input may be a voice input, a touch input, a gesture input, a click via a mouse or remote controller, and so on.

The mobile computing device 100 includes a display device 150 which presents information viewable by the user, for example on a user interface (e.g., a graphical user interface). For example, the display device 150 may be a non-touch sensitive display. The display device 150 may include a liquid crystal display (LCD), a light emitting diode (LED) display, an organic light emitting diode (OLED) display, active matrix organic light emitting diode (AMOLED), flexible display, 3D display, a plasma display panel (PDP), a cathode ray tube (CRT) display, and the like, for example. However, the disclosure is not limited to these example display devices and may include other types of display devices.

The mobile computing device 100 includes an output device 160 configured to provide an output to the user and may include, for example, one or more of an audio device (e.g., one or more speakers), a haptic device to provide haptic feedback to a user, a light source (e.g., one or more light sources such as LEDs which provide visual feedback to a user), and the like. For example, in some implementations of the disclosure the user may be guided through a process for taking a biometric measurement (e.g., via a virtual assistant 136 as shown in FIG. 3). The output device 160 may provide various indications to inform, alert, or notify the user to perform a certain action as part of the process for taking the biometric measurement. For example, in some implementations of the disclosure the user may be directed to move the mobile computing device 100 closer to the head of the user so as to be in contact with the head, the user may be given a countdown to indicate a duration of time for which the mobile computing device 100 is to be held against the head of the user, or the user may be provided with haptic feedback or a generic sound during a phone call to indicate that a biometric measurement is being taken. For example, the output device 160 may be configured to provide a warning, an alert, and/or a notification to the user in response to the one or more processors 110, 310 determining the biometric information of the user indicates the presence of an abnormality or an irregularity in the biometric information of the user. For example, in response to the one or more processors 110, 310 determining the biometric information of the user indicates the presence of an abnormality or an irregularity in the biometric information of the user, the mobile computing device 100 may be configured to communicate with one or more external computing devices 300 which may cause medication to be administered to the user. For example, the external computing device 300 may include a medical device (which may be attached to or implanted in the user) which can be remotely controlled (e.g., by the mobile computing device 100 or by another external computing device 300) to deliver medication (drugs) to the user in response to the one or more processors 110, 310 determining the biometric information of the user indicates the presence of the abnormality or the irregularity.

The mobile computing device 100 includes one or more cameras 170. For example, the one or more cameras 170 may include an imaging sensor (e.g., a complementary metal-oxide-semiconductor (CMOS) or charge-coupled device (CCD)) to capture, detect, or recognize a user's behavior, figure, expression, etc. In some implementations, the one or more cameras 170 may be used to detect (sense) a distance between the mobile computing device 100 and the head of the user. If the one or more cameras 170) detect that a distance between the mobile computing device and the head is less than a predetermined threshold (e.g., 1 cm to 3 cm), the mobile computing device 100 may be configured to execute or enable the BCG biometric measurement application 130 so that biometric information of the user can be obtained when the mobile computing device 100 is pressed against the head of the user. If the one or more cameras 170 detect that a distance between the mobile computing device 100 and the head is greater than the predetermined threshold, the mobile computing device 100 may be configured to terminate or disable the BCG biometric measurement application 130. In some implementations, a LIDAR may be used to measure a distance between the mobile computing device 100 and the head of the user.

The mobile computing device 100 includes one or more cameras 170. For example, the one or more cameras 170 may include an imaging sensor (e.g., a complementary metal-oxide-semiconductor (CMOS) or charge-coupled device (CCD)) to capture, detect, or recognize a user's behavior, figure, expression, etc. In some implementations, the one or more cameras 170 may be used to detect (sense) a distance between the mobile computing device 100 and the head of the user. If the one or more cameras 170 detect that a distance between the mobile computing device 100 and the head is less than a predetermined threshold (e.g., 1 cm to 3 cm), the mobile computing device 100 may be configured to execute or enable the BCG biometric measurement application 130 so that biometric information of the user can be obtained when the mobile computing device 100 is pressed against the head of the user. If the one or more cameras 170 detect that a distance between the mobile computing device 100 and the head is greater than the predetermined threshold, the mobile computing device 100 may be configured to terminate or disable the BCG biometric measurement application 130. In addition, the output device 160 may be configured to provide an indication to the user to move the mobile computing device 100 to be in contact with the head of the user (e.g., via a message played by a speaker, by a message presented on the display device 150, etc.).

The mobile computing device 100 includes one or more sensors 180. For example, the one or more sensors 180 may include an inertial measurement unit 182 which includes one or more accelerometers 182a and/or one or more gyroscopes 182b. The one or more accelerometers 182a may be used to capture motion information with respect to the mobile computing device 100. The one or more gyroscopes 182b may also be used additionally or alternatively to capture motion information with respect to the mobile computing device 100. For example, when the mobile computing device 100 is pressed against the head of the user (e.g., near a temple region of the head) the inertial measurement unit 182 may be configured to capture and amplify small above-skin motion signals caused by blood vessel volume changes for every pump of blood from the heart throughout the body of the user. For example, the inertial measurement unit 182 may be configured as a six-axis or six-dimensional inertial measurement unit (e.g., a tri-axial accelerometer and a tri-axial gyroscope). For example, the inertial measurement unit 182 may be configured to sample motions signals at a sample rate of 100 Hertz.

The one or more sensors 180 may also include a proximity sensor 184. For example, the proximity sensor 184 may be used to detect (sense) a distance between the mobile computing device 100 and the head of the user. If the proximity sensor 184 detects that a distance between the mobile computing device 100 and the head is less than a predetermined threshold (e.g., 1 cm to 3 cm), the mobile computing device 100 may be configured to execute or enable the BCG biometric measurement application 130 so that biometric information of the user can be obtained when the mobile computing device 100 is pressed against the head of the user. If the proximity sensor 184 detects that a distance between the mobile computing device 100 and the head is greater than the predetermined threshold, the mobile computing device 100 may be configured to terminate or disable the BCG biometric measurement application 130. In addition, the output device 160 may be configured to provide an indication to the user to move the mobile computing device 100 to be in contact with the head of the user (e.g., via a message played by a speaker, by a message presented on the display device 150, etc.).

The one or more sensors 180 may also include other sensors such as a magnetometer, GPS sensor, and the like. For example, in some implementations, a LIDAR may be used to measure a distance between the mobile computing device 100 and the head of the user.

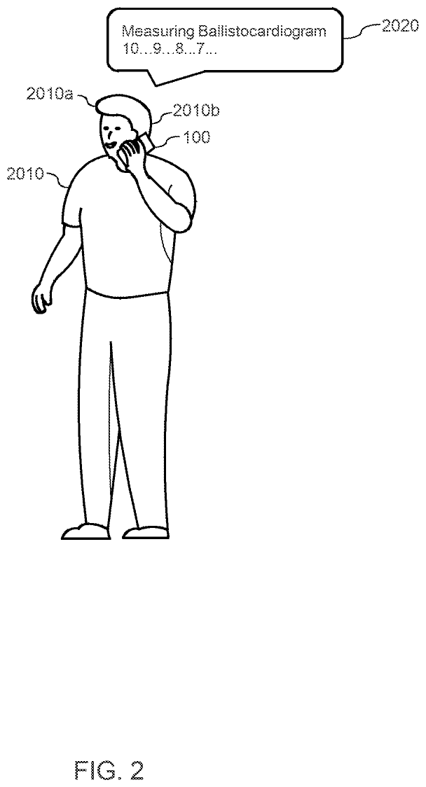

Referring to FIG. 2, an example illustration of a user holding a mobile computing device against their head is shown, according to one or more examples of the disclosure. In FIG. 2, the user 2010 is holding the mobile computing device 100 against their head 2010a, for example, near a temple region 2010b of the head 2010a.

In an example embodiment in which the user 2010 actively executes the BCG biometric measurement application 130, the virtual assistant 136 may be configured to instruct the user (via the output device 160) to hold the mobile computing device 100 against the user's head 2010a, for example, near the temple region 2010b of the head 2010a. The virtual assistant 136 may be configured to instruct the user (via the output device 160) to hold the mobile computing device 100 for a predetermined period of time (e.g., 20 seconds to 30 seconds). The virtual assistant 136 may be configured to notify the user (via the output device 160) that the biometric information is being measured (e.g., by playing a message 2020 with a countdown, by providing haptic feedback, etc.). The mobile computing device 100 may be configured to detect that the mobile computing device 100 is in contact with the head 2010a of the user 2010 based on an output provided by one or more of the proximity sensor 184, the one or more cameras 170, or another sensor such as a LIDAR, and the virtual assistant 136 may be configured to notify the user that the biometric information is being measured in response to the output indicating a distance between the mobile computing device 100 and the head 2010a being less than a predetermined distance (e.g., less than one centimeter).

In an example embodiment in which the BCG biometric measurement application 130 is automatically executed in response to the user 2010 making a phone call or receiving a phone call to passively measure biometric information of the user, the BCG biometric measurement application 130 may be configured to cause the output device 160 to provide an indication to the user 2010 (e.g., as haptic feedback through a haptic device or a sound output by a speaker that does not interrupt the phone call) which serves as a reminder that the mobile computing device 100 is taking a biometric measurement during the phone call. Therefore, the user may be more likely to hold the phone against their head during the phone call to obtain an accurate biometric measurement. The mobile computing device 100 may be configured to detect that the mobile computing device 100 is in contact with the head 2010a of the user 2010 based on an output provided by one or more of the proximity sensor 184, the one or more cameras 170, or another sensor such as a LIDAR, and the BCG biometric measurement application 130 may be configured to cause the output device 160 to notify the user that the biometric information is being measured in response to the output indicating a distance between the mobile computing device 100 and the head 2010a being less than a predetermined distance (e.g., less than one centimeter). In some implementations, if a phone call is less than a predetermined duration of time (e.g., less than 20 seconds to 30 seconds) or a measurement period during the phone call is less than the predetermined duration of time, the BCG biometric measurement application 130 may be configured to discard any data or biometric measurements obtained during the phone call less than the predetermined duration of time.

Referring to FIG. 3, an example block diagram of a biometric measurement application is shown, according to one or more examples of the disclosure. FIG. 3 illustrates that the BCG biometric measurement application 130 includes a ballistocardiographic autoencoder 132, spectral analyzer 134, and virtual assistant 136. However, the BCG biometric measurement application 130 may include fewer or more features than that shown in FIG. 3. For example, any of the ballistocardiographic autoencoder 132, spectral analyzer 134, and virtual assistant 136 may be provided separately from the BCG biometric measurement application 130. The BCG biometric measurement application 130 may access these separately provided features to perform operations or functions of the BCG biometric measurement application 130 such as measuring the biometric information of the user. For example, the BCG biometric measurement application 130 may be executed in an active manner or a passive manner as described previously.

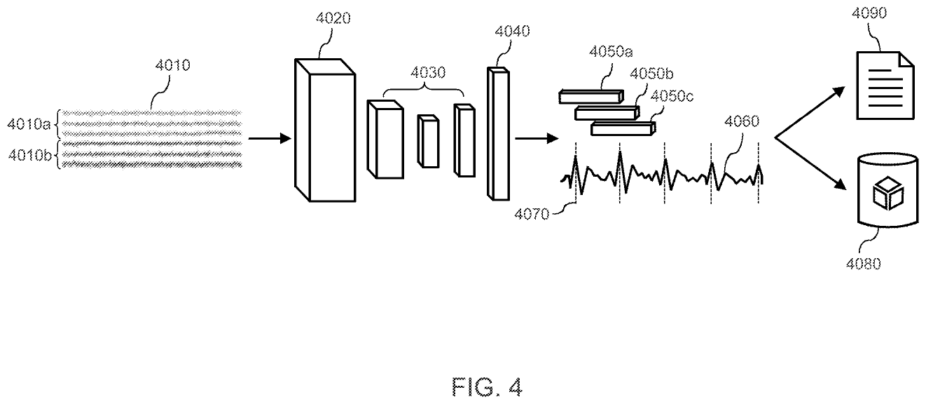

Operations of the BCG biometric measurement application 130 will now be described in more detail with reference to FIG. 3 and to FIG. 4 which illustrates an example flow diagram for obtaining biometric information of a user, according to one or more examples of the disclosure. For example, the mobile computing device 100 includes the IMU 182 which is configured to, when the mobile computing device 100 is pressed or held against the head of the user, detect and amplify small above-skin motion signals 4010 caused or generated by blood vessel volume changes for every pump of blood from the heart of the user throughout the body. The motion signals 4010 detected by the IMU 182 are transferred to the ballistocardiographic autoencoder 132 which is configured to output a realistic ballistocardiogram (BCG) signal 4060 based on the motion signals 4010 detected by the IMU 182. For example, the BCG signal 4060 may correspond to the small vibrations around the temple of the user which correlates to heart beats. That is, the BCG signal 4060 reflects a reaction (e.g., a displacement, velocity, and/or acceleration) of a part of the body resulting from cardiac ejection of blood. The spectral analyzer 134 is configured to analyze the BCG signal 4060 to output biometric information about the user, such as heart rate (HR), heart rate variability (HRV), etc. For example, the biometric information may be stored in a database 4080 of the mobile computing device 100 or of the one or more external computing devices 300, may be presented on the display device 150, for example in the form of a report 4090, may be stored in one or more of the memory devices 120, 320, etc.

In an example embodiment, the BCG biometric measurement application 130 may be automatically executed in response to the user 2010 making a phone call or receiving a phone call, to thereby passively measure biometric information of the user. For example, the one or more processors 110 of the mobile computing device 100 may be configured to recognize the initiation of a phone call or the reception of a phone call. For example, the one or more processors 110 of the mobile computing device 100 may be configured to automatically execute the BCG biometric measurement application 130 in response to recognizing the initiation of the phone call or the reception of the phone call. In some implementations, the one or more processors 110 of the mobile computing device 100 may be configured to automatically execute the BCG biometric measurement application 130 in response to recognizing the initiation of the phone call or the reception of the phone call and after the proximity sensor (or the one or more cameras 170) or some other sensor) detects that the mobile computing device 100 is in contact with the head of the user or is approaching the head of the user and is less than a predetermined distance from the head of the user (e.g., less than 3 cm).

In response to the telephone call being conducted and the BCG biometric measurement application 130 being automatically executed as discussed above, the one or more processors 110 may be configured to automatically perform the operations of: controlling the IMU 182 to detect the one or more motion signals, determining a ballistocardiogram signal (e.g., using the ballistocardiographic autoencoder 132) based on the one or more motion signals detected by the IMU 182, obtaining, based on the ballistocardiogram signal, biometric information of the user (e.g., using the spectral analyzer 134), and outputting the biometric information of the user. Accordingly, biometric information of the user is obtained and output in a passive manner during a telephone call conducted via the mobile computing device 100.

For example, as shown in FIG. 4, the motion signals 4010 measured by the IMU 182 may include a six-dimensional IMU signal that includes information from one or more accelerometers 182a (three-dimensions) and one or more gyroscopes 182b (three-dimensions). For example, the motion signals 4010 may be sampled by the IMU 182 at a sample rate of 100 Hertz, however the disclosure is not limited to this example sample rate. As shown in FIG. 4, the motion signals 4010 include three accelerometer signals 4010a which provide motion information about three axes, and three gyroscope signals 4010b which also provide motion information about three axes. For example, in some implementations the one or more accelerometers 182a may provide motion information about a single axis or two axes. For example, in some implementations the one or more gyroscopes 182b may provide motion information about a single axis or two axes. The motion signals 4010 measured by the IMU 182 may be transmitted to the ballistocardiographic autoencoder 132 (e.g., directly or via the one or more processors 110) to be converted to the BCG signal 4060 for cardiovascular analytics to be performed by the spectral analyzer 134. In some implementations, the motion signals 4010 measured by the IMU 182 may be transmitted to a ballistocardiographic autoencoder provided by the one or more external computing devices 300 to be converted to the BCG signal 4060. The one or more external computing devices 300 may also include a spectral analyzer (similar to spectral analyzer 134) to perform cardiovascular analytics with respect to the BCG signal 4060.

As illustrated in FIG. 3, the ballistocardiographic autoencoder 132 may include a first machine learning resource 132a which is configured to predict or generate a BCG signal (e.g., BCG signal 4060) via machine learning (e.g., via a custom neural network), based on the motion signals 4010. The ballistocardiographic autoencoder 132 is configured to convert the motion signals 4010 measured or output by the IMU 182 to the BCG signal 4060. For example, the ballistocardiographic autoencoder 132 may be configured to convert the motion signals 4010 to the BCG signal 4060 via a first machine learning resource 132a based on a mapping relationship between previously measured motion signals and corresponding BCG signals which are experimentally obtained (e.g., in a closed-room clinical setting) and used as training data for training the first machine learning resource 132a (e.g., an unsupervised neural network). For example, the ballistocardiographic autoencoder 132 may be configured to map the 6-dimensional IMU motion signals 4010 into the 1-dimensional BCG signal 4060. For example, the neural network may be trained so that biometric information obtained from the prediction of the BCG signal corresponds to biometric information obtained from the ground truth BCG signal (e.g., within a threshold level or tolerance, such as 5% or 10%).

In some implementations, the first machine learning resource 132a may be embodied as a custom neural network (e.g., an unsupervised neural network) and may include, as training data, IMU data (e.g., raw IMU motion signals) which is collected from subjects during a phone call session and corresponding ground truth BCG signal data collected from a BCG measurement device such as a piezoelectric sensor (e.g. a piezo band) which can be placed around the head or chest of the subject during the phone call session. In some implementations, other BCG measurement devices may be used to obtain a ground truth BCG signal such as a strain gauge, fiberoptic sensor, etc. The first machine learning resource 132a may be trained to predict or model a BCG signal (e.g., through regressive modeling) according to received IMU motion signals based on the training data and a machine learning algorithm that is derived from the training which occurs offline. Thus, a generalized mapping can occur between IMU motion signals (e.g., 6-dimensional IMU motion signals) obtained from an IMU and a 1-dimensional BCG signal using the first machine learning resource 132a of the ballistocardiographic autoencoder 132 to predict or generate the BCG signal 4060.

For example, as shown in FIG. 4, input data block 4020, intermediate data blocks 4030, and output data block 4040 represent a network architecture for an autoencoder including sequential operations (layers) of the first machine learning resource 132a. For example, input data block 4020 may correspond to a buffer input which corresponds to the motion signals 4010 (e.g., six-dimensional motion signals). For example, intermediate data blocks 4030 may correspond to encoder, bottleneck, and decoder layers (from left to right, respectively in FIG. 4) of the neural network. The encoder may be configured to produce a lower-dimensional encoding of the input data block 4020. The bottleneck layer may be a hidden layer which forces a learned compression of the input data block 4020 and the decoder may be configured to convert the original input data to a lower dimension (e.g., 1-dimension) output that corresponds to the BCG signal 4060.

The spectral analyzer 134 is configured to analyze spectral features of the BCG signal 4060 (e.g., 1-dimensional BCG signal) output by the ballistocardiographic autoencoder 132 to obtain biometric information of the user. For example, in some implementations the spectral analyzer 134 is configured to take a finite buffer of the BCG signal 4060, window the finite buffer in a manner to ensure infinity-spectral leakage does not occur, take a Fast Fourier transform of the BCG signal 4060 over the window, and perform some smoothed-variant of peak finding (e.g., using a peak detector which may be part of the spectral analyzer 134 or a separate feature of the BCG biometric measurement application 130) to locate a heart rate in beats per minute based on where a frequency has a maximum energy level in the frequency domain. For example, as shown in FIG. 4, a first finite buffer 4050a, a second finite buffer 4050b, and a third finite buffer 4050c correspond to portions of the BCG signal 4060 output by the ballistocardiographic autoencoder 132 over time which are windowed and which overlap with one another. The first finite buffer 4050a, second finite buffer 4050b, and third finite buffer 4050c can be combined together to form the BCG signal 4060. For example, the first finite buffer 4050a, second finite buffer 4050b, and third finite buffer 4050c can be concatenated to link the buffers together to form the BCG signal 4060 over time. Peaks 4070 of the BCG signal 4060 are indicated in FIG. 4 by the vertical dashed line passing through portions of the BCG signal 4060 having a greatest amplitude.

The spectral analyzer 134 may also be configured to analyze the BCG signal 4060 to ensure or verify that the obtained BCG signal 4060 is a “clean” signal which can provide accurate biometric information of the user. For example, the spectral analyzer 134 may be configured to determine whether the BCG signal 4060 satisfies one or more predetermined conditions before the spectral analyzer 134 proceeds with measuring the biometric information of the user from the BCG signal 4060. If the spectral analyzer 134 determines the BCG signal 4060 does not satisfy one or more of the one or more predetermined conditions, the spectral analyzer 134 may discard the data associated with that portion of the BCG signal 4060 and not measure the biometric information of the user from the BCG signal 4060.

In some implementations, a first predetermined condition may be that a noise level of the BCG signal 4060 is less than a threshold noise level. The spectral analyzer 134 may determine the first predetermined condition is not satisfied if the noise level of the BCG signal 4060) exceeds the threshold noise level.

In some implementations, a second predetermined condition may be that the user is stationary (at rest) and not moving. For example, when the user is moving the BCG signal 4060 may become contaminated with motion artifacts and biometric measurements of the BCG signal 4060 may be erroneous and/or inaccurate. The spectral analyzer 134 may determine the second predetermined condition is not satisfied if the user is moving (not at rest). The spectral analyzer 134 may determine the user is not at rest if the number of motion artifacts included in the BCG signal 4060 during a certain time period exceeds a threshold level. The spectral analyzer 134 may also determine the user is not at rest based on information received from other sensors of the mobile computing device 100 (e.g., from the one or more cameras 170) which indicate the user is not stationary.

In some implementations, a third predetermined condition may be that a sparsity level of the BCG signal 4060 is greater than a threshold sparsity level. For example, the sparsity level may be determined based on spectral features relating to peaks of the BCG signal 4060 relative to other portions of the BCG signal 4060. For example, the spectral analyzer 134 may determine the sparsity level based on a difference between a maximum energy level of the BCG signal 4060 and a median energy level of the BCG signal 4060. For example, the spectral analyzer 134 may determine the sparsity level is acceptable when the difference between the maximum energy level and the median energy level is greater than the threshold sparsity level, and unacceptable when the difference between the maximum energy level and the median energy level is less than the threshold sparsity level.

The spectral analyzer 134 may continuously perform checks of spectral features of the BCG signal 4060 which is passively obtained during a phone call (or actively obtained by a user intentionally requesting the biometric measurement) to determine whether the one or more predetermined conditions are satisfied, and when the one or more predetermined conditions are satisfied the spectral analyzer 134 may be configured to measure the biometric information of the user, for example by determining a heart rate and/or heart rate variability of the user based on the BCG signal 4060.

In some implementations, the spectral analyzer 134 may analyze the BCG signal 4060 to ensure or verify that the obtained BCG signal 4060 satisfies one or more predetermined conditions by using a second machine learning resource 134a. For example, the second machine learning resource 134a may be trained to determine whether the one or more predetermined conditions are satisfied by training a shallow; fully-connected neural network classifier on the spectral features of the BCG signal 4060 to perform a binary classification of: (1) signifying the one or more predetermined conditions are satisfied and it is a good time to proceed with measuring the biometric information of the user, or (2) signifying one or more of the one or more predetermined conditions are not satisfied and it is not a good time to proceed with measuring the biometric information of the user. The spectral analyzer 134 proceeds to measuring the biometric information of the user such as the heart rate or heart rate variability when the second machine learning resource 134a classifies the spectral features of the BCG signal according to the first classification. In some implementations, if the spectral analyzer 134 does not proceed with measuring the biometric information of the user when the second machine learning resource 134a classifies the spectral features of the BCG signal according to the second classification, the output device 160 of the mobile computing device 100 may be configured to provide an indication to the user. For example, if the spectral features of the BCG signal 4060 are classified according to the second classification due to movement of the user, the output device 160 may be configured to provide an indication to the user to remain still (e.g., via a speaker, display device, etc.). For example, if the spectral features of the BCG signal 4060 are classified according to the second classification due to excess noise and/or a low sparsity level, the output device 160 may be configured to provide an indication to the user to make another measurement attempt and/or to prompt the user to ensure the mobile computing device 100 is being held against the side of the head of the user (e.g., via a speaker, display device, etc.).

The spectral analyzer 134 is configured to measure or compute the biometric information of the user based on the BCG signal 4060 (e.g., in response to the BCG signal being classified according to the first classification). The spectral analyzer 134 may be configured to output the measured biometric information of the user, for example by storing the biometric information in a database, the one or more memory devices 120, the one or more memory devices 320, etc. In addition, or alternatively, the spectral analyzer 134 may be configured to output the measured biometric information of the user for presentation to the user on the display device 150. In some implementations, the biometric information may be displayed or provided to the user according to a time and/or date that the biometric information was obtained (e.g., by annotating a date of a phone call session). The BCG biometric measurement application 130 may be configured to generate a report which summarizes the obtained biometric information of the user. For example, the report may include a monthly summary, average resting heart parameters for the user, etc. For example, the report may include information such as a graph that is generated by interpolating (e.g., via exponential averaging and interpolation) the obtained biometric information of the user so that the user can view their heart rate as a continuous function of time.

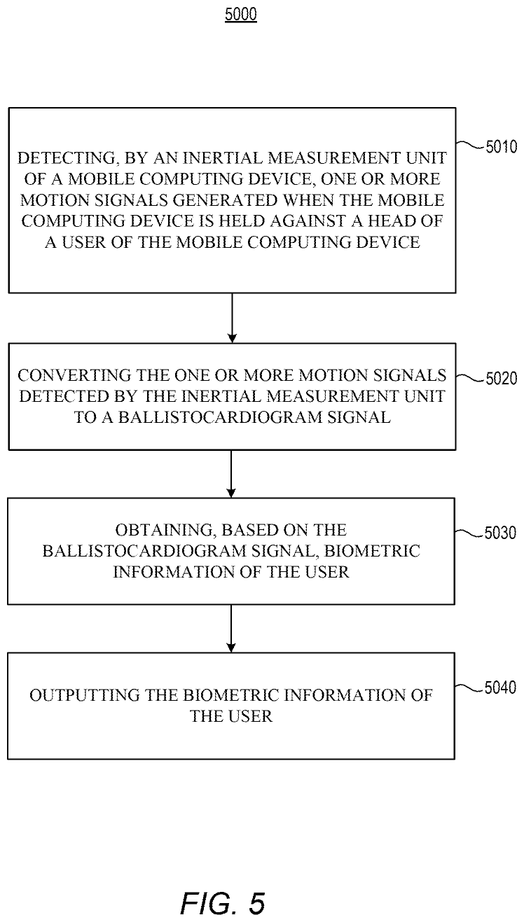

Referring to FIG. 5, a flow diagram of an example, non-limiting computer-implemented method according to one or more examples of the disclosure. The flow diagram FIG. 5 illustrates a method 5000 for obtaining biometric information of a user based on a BCG signal and for outputting the biometric information.

At 5010, the method includes detecting, by an IMU of a mobile computing device, one or more motion signals generated when the mobile computing device is held against a head of a user of the mobile computing device. For example, the IMU 182 is configured to capture and amplify small above-skin motion signals caused by blood vessel volume changes for every pump of blood from the heart throughout the body of the user when the mobile computing device 100 is held against the head of the user.

At 5020, the method includes converting the one or more motion signals detected by the IMU to a ballistocardiogram signal. For example, the IMU 182 is configured to output or transmit the one or motion signals to the ballistocardiographic autoencoder 132. The ballistocardiographic autoencoder 132 is configured to convert the one or more motion signals to the BCG signal, for example, via a first machine learning resource 132a.

At 5030, the method includes obtaining, based on the ballistocardiogram signal, biometric information of the user. For example, the ballistocardiographic autoencoder 132 is configured to output or transmit the BCG signal to the spectral analyzer 134. The spectral analyzer 134 is configured to analyze spectral features of the BCG signal to obtain the biometric information of the user. The spectral analyzer 134 may also be configured to analyze the BCG signal to ensure or verify that the obtained BCG signal is a “clean” signal which can provide accurate biometric information of the user.

At 5040, the method includes outputting the biometric information of the user. For example, the spectral analyzer 134 may be configured to output the biometric information of the user, for example by storing the biometric information in a database, the one or more memory devices 120, the one or more memory devices 320, etc. In addition, or alternatively, the spectral analyzer 134 may be configured to output the biometric information of the user for presentation to the user on the display device 150.

Aspects of the above-described example embodiments may be recorded in non-transitory computer-readable media including program instructions to implement various operations embodied by a computer. The media may also include, alone or in combination with the program instructions, data files, data structures, and the like. Examples of non-transitory computer-readable media include magnetic media such as hard disks, floppy disks, and magnetic tape: optical media such as CD ROM disks, Blue-Ray disks, and DVDs: magneto-optical media such as optical discs: and other hardware devices that are specially configured to store and perform program instructions, such as semiconductor memory, read-only memory (ROM), random access memory (RAM), flash memory, USB memory, and the like. Examples of program instructions include both machine code, such as produced by a compiler, and files containing higher level code that may be executed by the computer using an interpreter. The program instructions may be executed by one or more processors. The described hardware devices may be configured to act as one or more software modules in order to perform the operations of the above-described embodiments, or vice versa. In addition, a non-transitory computer-readable storage medium may be distributed among computer systems connected through a network and computer-readable codes or program instructions may be stored and executed in a decentralized manner. In addition, the non-transitory computer-readable storage media may also be embodied in at least one application specific integrated circuit (ASIC) or Field Programmable Gate Array (FPGA).

Each block of the flowchart illustrations may represent a unit, module, segment, or portion of code, which comprises one or more executable instructions for implementing the specified logical function(s). It should also be noted that in some alternative implementations, the functions noted in the blocks may occur out of order. For example, two blocks shown in succession may in fact be executed substantially concurrently (simultaneously) or the blocks may sometimes be executed in the reverse order, depending upon the functionality involved.

While the disclosure has been described with respect to various example embodiments, each example is provided by way of explanation, not limitation of the disclosure. Those skilled in the art, upon attaining an understanding of the foregoing, can readily produce alterations to, variations of, and equivalents to such embodiments. Accordingly, the disclosure does not preclude inclusion of such modifications, variations and/or additions to the disclosed subject matter as would be readily apparent to one of ordinary skill in the art. For example, features illustrated or described as part of one embodiment can be used with another embodiment to yield a still further embodiment. Thus, it is intended that the disclosure covers such alterations, variations, and equivalents.

What is claimed is:

1. A mobile computing device, comprising:
one or more memories configured to store one or more instructions:
an inertial measurement unit: and one or more processors configured to execute the one or more instructions stored in the one or more memories to:
control the inertial measurement unit to detect one or more motion signals generated when the mobile computing device is held against a head of a user of the mobile computing device,
determine a ballistocardiogram signal based on the one or more motion signals detected by the inertial measurement unit,
obtain, based on the ballistocardiogram signal, biometric information of the user, and
output the biometric information of the user.

2. The mobile computing device of claim 1, wherein:
the one or more processors are configured to automatically execute the one or more instructions stored in the one or more memories to control the inertial measurement unit to detect the one or more motion signals, in response to a telephone call being conducted using the mobile computing device.

3. The mobile computing device of claim 2, wherein the one or more motion signals are generated based on blood vessel volume changes in a temple region of the head of the user.

4. The mobile computing device of claim 2, further comprising an output device configured to provide, during the telephone call, an indication that the mobile computing device is performing a process to obtain the biometric information of the user.

5. The mobile computing device of claim 4, wherein the indication includes at least one of a sound provided by the output device or haptic feedback provided by the output device.

6. The mobile computing device of claim 1, further comprising:
a ballistocardiographic autoencoder; and
a spectral analyzer,
wherein
the one or more processors are configured to determine the ballistocardiogram signal by controlling the ballistocardiographic autoencoder to convert the one or more motion signals detected by the inertial measurement unit to the ballistocardiogram signal, and
the one or more processors are configured to obtain the biometric information of the user by controlling the spectral analyzer to:
perform a Fast Fourier transform with respect to the ballistocardiogram signal over a predetermined window of the ballistocardiogram signal,
detect peaks, with respect to the ballistocardiogram signal, during the predetermined window, and
obtain the biometric information of the user based on the peaks which are detected during the predetermined window.

7. The mobile computing device of claim 6, wherein:
the ballistocardiographic autoencoder is configured to convert the one or more motion signals detected by the inertial measurement unit to the ballistocardiogram signal using a machine learning resource which predicts the ballistocardiogram signal based on training data that maps a relationship between previous motion signals and corresponding ground truth ballistocardiogram signals.

8. The mobile computing device of claim 6, wherein:
the spectral analyzer is configured to determine whether the ballistocardiogram signal satisfies one or more predetermined conditions before determining the biometric information of the user based on the ballistocardiogram signal, and
the one or more predetermined conditions are associated with at least one of a noise level of the ballistocardiogram signal, a motion of the user, or a sparsity level of the ballistocardiogram signal.

9. The mobile computing device of claim 8, wherein:
when a first predetermined condition among the one or more predetermined conditions relates to the noise level of the ballistocardiogram signal, the spectral analyzer is configured to determine the ballistocardiogram signal satisfies the first predetermined condition when the noise level associated with the ballistocardiogram signal is less than a threshold noise level,
when a second predetermined condition among the one or more predetermined conditions relates to the motion of the user, the spectral analyzer is configured to determine the ballistocardiogram signal satisfies the second predetermined condition when the user is determined to be at rest, and
when a third predetermined condition among the one or more predetermined conditions relates to the sparsity level of the ballistocardiogram signal, the spectral analyzer is configured to determine the ballistocardiogram signal satisfies the third predetermined condition when the sparsity level of the ballistocardiogram signal is greater than a threshold sparsity level.

10. The mobile computing device of claim 9, wherein the spectral analyzer is configured to:
determine a difference between a maximum energy level of the ballistocardiogram signal and a median energy level of the ballistocardiogram signal during the predetermined window, and
determine the ballistocardiogram signal satisfies the third predetermined condition when the difference between the maximum energy level and the median energy level is greater than the threshold sparsity level.

11. The mobile computing device of claim 6, wherein:
the inertial measurement unit includes one or more accelerometers and one or more gyroscopes to detect the one or more motion signals generated when the mobile computing device is held against the head of the user,
the one or more motion signals include a six-dimensional motion signal, and
the ballistocardiographic autoencoder is configured to convert the six-dimensional motion signal to a one-dimensional ballistocardiogram signal.

12. The mobile computing device of claim 1, wherein the mobile computing device is a mobile phone or a smartphone.

13. The mobile computing device of claim 1, wherein the biometric information of the user includes at least one of a heart rate of the user or a heart rate variability of the user.

14. The mobile computing device of claim 1, wherein the one or more processors are configured to output the biometric information of the user by at least one of:
storing the biometric information of the user in at least one of a database, the one or more memories, or one or more memories of an external computing device,
presenting the biometric information of the user on a display of the mobile computing device, or
generating a report which summarizes the biometric information of the user.

15. The mobile computing device of claim 1, further comprising an output device,
wherein the one or more processors are configured to analyze the biometric information of the user to determine whether the biometric information of the user indicates a presence of an abnormality or an irregularity in the biometric information of the user, and
the one or more processors are configured to control the output device to provide at least one of a warning, an alert, or a notification to the user in response to determining the biometric information of the user indicates the presence of the abnormality or the irregularity in the biometric information of the user.

16. The mobile computing device of claim 1, further comprising:
an input device configured to receive a request from the user to obtain the biometric information of the user; and
an output device configured to provide information regarding a measurement time period for which the mobile computing device is to be held against the head of the user of the mobile computing device.

17. A computer-implemented method, comprising:
detecting, by an inertial measurement unit of a mobile computing device, one or more motion signals generated when the mobile computing device is held against a head of a user of the mobile computing device:
converting the one or more motion signals detected by the inertial measurement unit to a ballistocardiogram signal:
obtaining, based on the ballistocardiogram signal, biometric information of the user; and
outputting the biometric information of the user.

18. The computer-implemented method of claim 17, further comprising automatically controlling the inertial measurement unit to detect the one or more motion signals in response to a telephone call being conducted using the mobile computing device.

19. The computer-implemented method of claim 17, wherein converting the one or more motion signals detected by the inertial measurement unit to the ballistocardiogram signal includes using a machine learning resource which predicts the ballistocardiogram signal based on training data that maps a relationship between previous motion signals and corresponding ground truth ballistocardiogram signals.

20. A non-transitory computer-readable medium which stores instructions that are executable by one or more processors of a mobile computing device, the instructions comprising:
instructions to cause the one or more processors to control an inertial measurement unit of the mobile computing device to detect one or more motion signals generated when the mobile computing device is held against a head of a user of the mobile computing device:
instructions to cause the one or more processors to convert the one or more motion signals detected by the inertial measurement unit to a ballistocardiogram signal:
instructions to cause the one or more processors to obtain, based on the ballistocardiogram signal, biometric information of the user; and
instructions to cause the one or more processors to output the biometric information of the user.
